(19) United States
(12) Patent Application Publication
(10) Pub. No.: US 2009/0180979 A1
(43) Pub. Date: Jul. 16, 2009

(54) COMPOSITION FOR THE OXIDATION DYEING OF KERATIN FIBERS COMPRISING AT LEAST ONE CELLULOSE WITH HYDROPHOBIC SUBSTITUENTS, AT LEAST ONE OXIDATION DYE, AND AT LEAST ONE LONG-CHAIN FATTY ALCOHOL, AND METHODS OF USE THEREOF

(76) Inventor: Marie-Pascale AUDOUSSET, Asnieres (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/267,741

(22) Filed: Nov. 10, 2008

Related U.S. Application Data
(60) Provisional application No. 60/996,463, filed on Nov. 19, 2007.

(30) Foreign Application Priority Data
Nov. 9, 2007 (FR) 0758930

(51) Int. Cl. A61K 8/34; A61Q 5/10

(52) U.S. Cl. 424/70.1

(57) ABSTRACT

The present disclosure relates to a dye composition for keratin fibers, comprising, in a medium suitable for dyeing: at least one nonionic derivative of cellulose comprising at least one hydrophobic substituent containing from 8 to 30 carbon atoms; at least one oxidation dye, and at least one fatty alcohol containing at least 20 carbon atoms.

This application claims benefit of U.S. Provisional Application No. 60/996,463, filed Nov. 19, 2007, the contents of which are incorporated herein by reference. This application also claims benefit of priority under 35 U.S.C. § 119 to French Patent Application No. FR 07 58930, filed Nov. 9, 2007, the contents of which are also incorporated herein by reference.

Disclosed herein is a composition for the oxidation dyeing of keratin fibers.

It is known practice to dye keratin fibers, including human hair, with dye compositions comprising oxidation dye precursors, generally known as oxidation bases, such as ortho- or para-phenylenediamines, ortho- or para-aminophenols and heterocyclic compounds. These oxidation bases are colorless or weakly colored compounds which, in combination with oxidizing products, can give rise, via an oxidative condensation process, to colored compounds.

It is also known that it is possible to vary the shades obtained with these oxidation bases by combining them with couplers or coloring modifiers, the latter being chosen for example, from aromatic meta-diamines, meta-aminophenols, meta-diphenols and certain heterocyclic compounds such as indole compounds.

The variety of the molecules involved as oxidation bases and couplers can make it possible to obtain a rich palette of colors.

The “permanent” coloring obtained by virtue of these oxidation dyes should, moreover, meet a certain number of requirements.

For example, it should have no toxicological drawbacks, it should allow shades to be obtained in the desired strength, and it should show good color-fastness with respect to external agents such as light, bad weather, washing, permanent-waving, perspiration, and rubbing.

In addition, the dyes should also allow white hair to be covered and, finally, should be as nonselective as possible, i.e. they should make it possible to obtain the smallest possible differences in coloring along the same keratin fiber, which is generally differently sensitized (i.e. damaged) between its tip and its root.

Moreover, the compositions obtained should, in addition, have good rheological properties, while at the same time conserve good coloring properties. For instance, these compositions should not run on the face or out of the areas intended to be dyed, when they are applied, such as after mixing with an oxidizing agent.

Improving the power of dyeing by combining a para-phenylenediamine oxidation base and at least one nonionic amphiphilic polymer such as hydroxycellulose modified with a hydrophobic group is discussed in International Patent Application No. WO 98/03150.

However, these compositions do not entirely meet the abovementioned requirements and can be improved, in terms of dyeing properties, for instance in terms of dyeing selectivity and power. Thus, there is a need in the art to obtain stable hair dyeing compositions, such as, in the form of creams, which are easy to prepare and to apply, which may contain high concentrations of dyes in the form of salts, which have good rheological qualities and which produce strong, relatively nonselective colorations that withstand the various attacks that keratin fibers may be subjected to.

Accordingly, one aspect of the present disclosure is a dye composition for keratin fibers, including human keratin fibers, such as the hair, that meets at least one of the conditions discussed above, comprising, in a medium suitable for dyeing:

(A) at least one nonionic derivative of cellulose comprising at least one hydrophobic substituent containing from 8 to 30 carbon atoms, present in an amount ranging from 0.1% to 1% by weight, relative to the total weight of the composition;

(B) at least one oxidation dye; and

(C) at least one fatty alcohol containing at least 20 carbon atoms.

The dye compositions according to the present disclosure can have at least one of the following properties:

they make it possible to obtain compositions with a viscosity of a cream, which are stable over time,

they stand out by virtue of the fact that they could be easily mixed with the oxidizing composition,

they stand out by virtue of the rheological qualities of the creams obtained (good viscosity of cream as a mixture),

they are easy to apply after mixing with the oxidizing composition at the time the dyeing is carried out (qualities of use on the head).

In addition, the compositions according to the present disclosure can make it possible to obtain compositions capable of producing colorings with varied, chromatic, powerful, aesthetic, and relatively nonselective shades which are uniform over all the keratin fibers, including human keratin fibers, such as the hair, and which are highly resistant to the various attacks to which the fibers may be subjected.

Another aspect of the present disclosure comprises a process for the dyeing of keratin fibers, wherein the cosmetic composition according to the present disclosure is used.

An additional aspect of the present disclosure relates to the use of this cosmetic composition for dyeing keratin fibers, including human keratin fibers, such as the hair.

Other features, aspects, subjects and benefits of the present disclosure will emerge more clearly on reading the description and the non-limiting examples which follow.

Unless otherwise indicated, the limits of the ranges of values which are given in the context of the present disclosure are included in these ranges.

As used herein, the term “derivative(s) of cellulose” is intended to mean at least one compound comprising at least one cellobiose unit having the following structure:

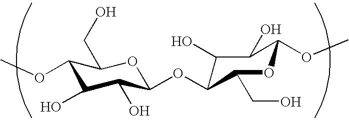

wherein at least one hydroxyl group may be substituted.

The at least one nonionic derivative of cellulose with at least one hydrophobic substituent (A) in accordance with the present disclosure is an amphiphilic polymer that is associative in nature. It comprises hydrophilic units and hydrophobic units and is capable of interacting and of associating with one another or with other molecules, reversibly, for instance, by virtue of the presence of their hydrophobic chains.

In at least one embodiment, the at least one cellulose derivative of the present disclosure is a cellulose ether comprising at least one hydrophobic substituent containing from 8 to 30 carbon atoms.

The at least one nonionic derivative of cellulose with at least one hydrophobic substituent in accordance with the present disclosure can be prepared from water-soluble nonionic ethers of cellulose, wherein all or some of the reactive hydroxyl functional groups are substituted with at least one hydrophobic chain containing from 8 to 30 carbon atoms, for example, containing 10 to 22 carbon atoms, and further for example, containing 16 carbon atoms. The reaction steps involved in the preparation of the at least one cellulose derivative of the present disclosure are known to those skilled in the art.

In at least one embodiment, the nonionic ethers of cellulose chosen for preparing the at least one nonionic derivative of cellulose with at least one hydrophobic substituent, according to the present disclosure, have a degree of nonionic substitution, for example of methyl, hydroxyethyl, or hydroxypropyl group(s), that is sufficient to be water-soluble, i.e. to form a substantially clear solution when dissolved in water at 25° C. at the concentration of 1% by weight.

The nonionic ethers of cellulose chosen for preparing the at least one nonionic derivatives of cellulose with at least one hydrophobic substituent according to the present disclosure, can have, for example, a relatively low number-average molar mass, such as less than 800,000 g/mol, for example ranging from 50,000 to 700,000 g/mol, and further for example, ranging from 200,000 to 600,000 g/mol.

In at least one embodiment, the at least one nonionic cellulose derivative of the present disclosure is a hydroxyethylcellulose comprising at least one hydrophobic substituent containing from 8 to 30 carbon atoms.

The at least one nonionic derivative of cellulose according to the present disclosure is substituted with at least one entity chosen from aliphatic and aromatic, saturated and unsaturated, linear, branched, and cyclic C₈-C₃₀ hydrocarbon chains, that may be attached to the cellulose ether substrate via an ether, ester, or urethane bond, and in at least one embodiment of the present disclosure, via an ether bond.

According to at least one embodiment, the hydrophobic substituents used as the at least one hydrophobic substituent of the at least one nonionic derivative of cellulose according to the present disclosure are chosen from C₈-C₃₀, for example, C₁₀-C₂₂, alkyl, arylalkyl, and alkylaryl groups.

For example, the at least one hydrophobic substituent according to the present disclosure can be chosen from saturated alkyl chains.

According to at least one embodiment, the at least one hydrophobic substituent according to the present disclosure is chosen from cetyl groups.

The at least one nonionic derivative of cellulose with at least one hydrophobic substituent according to the disclosure has a viscosity ranging from 100 and 100,000 mPa·s, for example ranging from 200 and 20,000 mPa·s, measured at 25° C. in a solution at 1% by weight of polymer in water, this viscosity being determined conventionally using a Brookfield LVT viscometer at 6 rpm with the No. 3 spindle.

The degree of hydrophobic substitution of the hydrophilic nonionic derivatives of cellulose used according to the present disclosure can range, for example, from 0.1% to 10% by weight, for example from 0.1% to 1% by weight, and further for example from 0.4% to 0.8% by weight, relative to the total weight of the polymer.

Non-limiting examples of the at least one nonionic derivative of cellulose with at least one hydrophobic substituent of the present disclosure include cetyl hydroxyethylcelluloses sold, for instance, under the names NATROSOL® Plus Grade 330 CS and POLYSURF® 67 CS (INCl: Cetyl Hydroxyethylcellulose) by the company Aqualon/Hercules.

The at least one oxidation dye (B) according to the present disclosure may be chosen from oxidation bases and oxidation couplers.

The at least one oxidation dye (B) according to the present disclosure is, in at least one embodiment, chosen from oxidation bases, oxidation couplers, and addition salts thereof.

Non-limiting examples of oxidation bases that can be used include para-phenylenediamines, bisphenylalkylenediamines, para-aminophenols, bis-para-aminophenols, ortho-aminophenols, heterocyclic bases, and addition salts thereof.

Among the para-phenylenediamines, non-limiting mention may be made, for example, of para-phenylenediamine; para-toluoylenediamine; 2-chloro-para-phenylenediamine; 2,3-dimethyl-para-phenylenediamine; 2,6-dimethyl-para-phenylenediamine; 2,6-diethyl-para-phenylenediamine; 2,5-dimethyl-para-phenylenediamine; N,N-dimethyl-para-phenylenediamine; N,N-diethyl-para-phenylene-diamine; N,N-dipropyl-para-phenylenediamine; 4-amino-N,N-diethyl-3-methylaniline; N,N-bis(β-hydroxyethyl)-para-phenylenediamine; 4-N,N-bis(β-hydroxyethyl)amino-2-methylaniline; 4-N,N-bis(β-hydroxyethyl)amino-2-chloroaniline; 2-β-hydroxyethyl-para-phenylenediamine; 2-fluoro-para-phenylenediamine; 2-isopropyl-para-phenylenediamine; N-(β-hydroxypropyl)-para-phenylenediamine; 2-hydroxymethyl-para-phenylenediamine; N,N-dimethyl-3-methyl-para-phenylenediamine; N,N-(ethyl-p-hydroxyethyl)-para-phenylenediamine; N-(β,β-dihydroxypropyl)-para-phenylenediamine; N-(4′-aminophenyl)-para-phenylenediamine; N-phenyl-para-phenylenediamine; 2-β-hydroxyethyloxy-para-phenylenediamine; 2-β-acetylaminoethyloxy-para-phenylenediamine; N-(β-methoxyethyl)-para-phenylenediamine; 4-aminophenylpyrrolidine; 2-thienyl-para-phenylenediamine; 2-β-hydroxyethylamino-5-aminotoluene; 3-hydroxy-1-(4′-aminophenyl)pyrrolidine; and the acid addition salts thereof.

Among the para-phenylenediamines mentioned above, further non-limiting mention may be made, for example, of para-phenylenediamine; para-toluoylenediamine; 2-isopropyl-para-phenylenediamine; 2-p-hydroxyethyl-para-phenylenediamine; 2-β-hydroxyethyloxy-para-phenylenediamine; 2,6-dimethyl-para-phenylenediamine; 2,6-diethyl-para-phenylenediamine; 2,3-dimethyl-para-phenylenediamine; N,N-bis(β-hydroxyethyl)-para-phenylenediamine; 2-chloro-para-phenylenediamine; 2-β-acetylaminoethyloxy-para-phenylenediamine; and acid addition salts thereof.

Among the bisphenylalkylenediamines, non-limiting mention may be made, for example, of N,N′-bis(β-hydroxyethyl)-N,N′-bis(4′-aminophenyl)-1,3-diaminopropanol; N,N′-bis(p-hydroxyethyl)-N,N′-bis(4′-aminophenyl)ethylenediamine; N,N′-bis(4-amino-phenyl)tetramethylenediamine; N,N′-bis(β-hydroxyethyl)-N,N′-bis(4-aminophenyl)tetra-methylenediamine; N,N′-bis-(4-methylaminophenyl)tetramethylenediamine; N,N′-bis(ethyl)-N,N′-bis(4′-amino-3′-methylphenyl)ethylenediamine; 1,8-bis(2,5-diaminophenoxy)-3,6-dioxaoctane; and the acid addition salts thereof.

Among the para-aminophenols, non-limiting mention may be made, for example, of para-aminophenol; 4-amino-3-methylphenol; 4-amino-3-fluorophenol; 4-amino-3-hydroxymethylphenol; 4-amino-2-methylphenol; 4-amino-2-hydroxymethylphenol; 4-amino-2-methoxymethylphenol; 4-amino-2-aminomethylphenol; 4-amino-2-(β-hydroxy-ethylaminomethyl)phenol; 4-amino-2-fluorophenol; and the acid addition salts thereof.

Among the ortho-aminophenols, non-limiting mention may be made, for example, of 2-aminophenol; 2-amino-5-methylphenol; 2-amino-6-methylphenol; 5-acetamido-2-aminophenol; and the acid addition salts thereof.

Among the heterocyclic bases, non-limiting mention may be made, for example, of pyridine derivatives, pyrimidine derivatives, pyrazole derivatives, pyrazolone derivatives, and addition salts thereof.

Among the pyridine derivatives, non-limiting mention may be made, for example, of 2,5-diaminopyridine; 2-(4-methoxyphenyl)amino-3-aminopyridine; 2,3-diamino-6-methoxypyridine; 2-(β-methoxyethyl)amino-3-amino-6-methoxypyridine; 3,4-diamino-pyridine; and addition salts thereof with an acid. Such pyridine derivatives are described, for example, in United Kingdom Patent Nos. GB 1,026,978 and GB 1,153,196.

Other non-limiting examples of pyridine oxidation bases that can be used in the present disclosure are the 3-aminopyrazolo[1,5-a]pyridine oxidation bases and addition salts thereof described, for example, in French Patent Application FR 2 801 308. By way of non-limiting example, mention may be made of pyrazolo[1,5-a]pyridin-3-ylamine; 2-acetylaminopyrazolo[1,5-a]pyridin-3-ylamine; 2-morpholin-4-ylpyrazolo[1,5-a]pyridin-3-ylamine; 3-aminopyrazolo[1,5-a]pyridin-2-carboxylic acid; 2-methoxypyrazolo[1,5-a]pyridin-3-ylamino; (3-aminopyrazolo[1,5-a]pyridin-7-yl)methanol; 2-(3-aminopyrazolo[1,5-a]pyridin-5-yl)ethanol; 2-(3-aminopyrazolo[1,5-a]pyridin-7-yl)ethanol; (3-aminopyrazolo[1,5-a]pyridin-2-yl)methanol; 3,6-diaminopyrazolo[1,5-a]pyridine; 3,4-diaminopyrazolo[1,5-a]pyridine; pyrazolo[1,5-a]pyridine-3,7-diamine; 7-morpholin-4-ylpyrazolo[1,5-a]pyridin-3-ylamine; pyrazolo[1,5-a]pyridine-3,5-diamine; 5-morpholin-4-ylpyrazolo[1,5-a]pyridin-3-ylamine; 2-[(3-aminopyrazolo[1,5-a]pyridin-5-yl)(2-hydroxyethyl)amino]ethanol; 2-[(3-amino-pyrazolo[1,5-a]pyridin-7-yl)(2-hydroxyethyl)amino]ethanol; 3-aminopyrazolo[1,5-a]pyridin-5-ol; 3-aminopyrazolo[1,5-a]pyridin-4-ol; 3-aminopyrazolo[1,5-a]pyridin-6-ol; 3-amino-pyrazolo[1,5-a]pyridin-7-ol; and also addition salts thereof with an acid or with a base.

Non-limiting mention may be made among the pyrimidine derivatives, of the compounds described, for example, in German Patent No. DE 2359399; Japanese Patent Nos. JP 88-169571 and JP 05-63124; European Patent No. EP 0770375; or International Patent Application No. WO 96/15765; such as 2,4,5,6-tetraminopyrimidine; 4-hydroxy-2,5,6-triaminopyrimidine; 2-hydroxy-4,5,6-triaminopyrimidine; 2,4-dihydroxy-5,6-diaminopyrimidine; 2,5,6-triaminopyrimidine; and pyrazolopyrimidine derivatives such as those mentioned in French Patent Application No. FR-A-2 750 048, and among which non-limiting mention may be made of pyrazolo[1,5-a]pyrimidine-3,7-diamine; 2,5-dimethyl-pyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol; 2-(7-aminopyrazolo[1,5-a]pyrimidin-3-ylamino)ethanol; 2-[(3-aminopyrazolo[1,5-a]pyrimidin-7-yl)(2-hydroxyethyl)amino]ethanol; 2-[(7-aminopyrazolo[1,5-a]pyrimidin-3-yl)(2-hydroxyethyl)amino]ethanol; 5,6-dimethylpyrazolo[1,5-a]pyrimidine-3,7-diamine; 2,6-dimethylpyrazolo[1,5-a]pyrimidine-3,7-diamine; 2,5,N7,N7-tetramethylpyrazolo[1,5-a]pyrimidine-3,7-diamine; 3-amino-5-methyl-7-imidazolyipropylaminopyrazolo[1,5-a]pyrimidine; and addition salts thereof with an acid and tautomeric forms thereof when a tautomeric equilibrium exists.

Among the pyrazole derivatives that can be used, non-limiting mention may be made, for example, of the compounds described in German Patent Nos. DE-A-38 43 892 and DE-A-41 33 957; German Patent Application No. DE-A-195 43 988; International Patent Application Nos. WO 94/08969 and WO 94/08970; and French Patent Application No. FR-A-2 733 749; such as 4,5-diamino-1-methylpyrazole; 4,5-diamino-1-(β-hydroxyethyl)pyrazole; 3,4-diaminopyrazole; 4,5-diamino-1-(4′-chlorobenzyl)pyrazole; 4,5-diamino-1,3-dimethylpyrazole; 4,5-diamino-3-methyl-1-phenylpyrazole; 4,5-diamino-1-methyl-3-phenylpyrazole; 4-amino-1,3-dimethyl-5-hydrazinopyrazole; 1-benzyl-4,5-diamino-3-methylpyrazole; 4,5-diamino-3-tert-butyl-1-methylpyrazole; 4,5-diamino-1-tert-butyl-3-methylpyrazole; 4,5-diamino-1-(β-hydroxyethyl)-3-methylpyrazole; 4,5-diamino-1-ethyl-3-methylpyrazole; 4,5-diamino-1-ethyl-3-(4′-methoxyphenyl)pyrazole; 4,5-diamino-1-ethyl-3-hydroxymethylpyrazole; 4,5-diamino-3-hydroxymethyl-1-methylpyrazole; 4,5-diamino-3-hydroxymethyl-1-isopropylpyrazole; 4,5-diamino-3-methyl-1-isopropylpyrazole; 4-amino-5-(2′-aminoethyl)amino-1,3-dimethylpyrazole; 3,4,5-triaminopyrazole; 1-methyl-3,4,5-triaminopyrazole; 3,5-diamino-1-methyl-4-methylaminopyrazole; 3,5-diamino-4-(β-hydroxyethyl)amino-1-methylpyrazole; and addition salts thereof.

Among the pyrazolone derivatives that can be used, non-limiting mention may, for example, be made of the following compounds and addition salts thereof:
2,3-diaminodihydropyrazolone;
4,5-diamino-1,2-dimethyl-1,2-dihydropyrazol-3-one;
4-amino-5-methylamino-1,2-dimethyl-1,2-dihydropyrazol-3-one;
4-amino-5-dimethylamino-1,2-dimethyl-1,2-dihydropyrazol-3-one;
4-amino-5-(2-hydroxyethyl)amino-1,2-dimethyl-1,2-dihydropyrazol-3-one;
4-amino-5-(pyrrolidin-1-yl)-1,2-dimethyl-1,2-dihydropyrazol-3-one;
4-amino-5-(piperidin-1-yl)-1,2-dimethyl-1,2-dihydropyrazol-3-one;
4,5-diamino-1,2-di(2-hydroxyethyl)-1,2-dihydropyrazol-3-one;
4-amino-5-methylamino-1,2-di(2-hydroxyethyl)-1,2-dihydropyrazol-3-one;
4-amino-5-dimethylamino-1,2-di(2-hydroxyethyl)-1,2-dihydropyrazol-3-one;
4-amino-5-(2-hydroxyethyl)amino-1,2-di(2-hydroxyethyl)-1,2-dihydropyrazol-3-one;
4-amino-5-(pyrrolidin-1-yl)-1,2-di(2-hydroxyethyl)-1,2-dihydropyrazol-3-one;
4-amino-5-(piperidin-1-yl)-1,2-di(2-hydroxyethyl)-1,2-dihydropyrazol-3-one;
4,5-diamino-1,2-diethyl-1,2-dihydropyrazol-3-one;
4,5-diamino-1,2-diphenyl-1,2-dihydropyrazol-3-one;
4,5-diamino-1-ethyl-2-methyl-1,2-dihydropyrazol-3-one;
4,5-diamino-2-ethyl-1-methyl-1,2-dihydropyrazol-3-one;
4,5-diamino-1-phenyl-2-methyl-1,2-dihydropyrazol-3-one;
4,5-diamino-2-phenyl-1-methyl-1,2-dihydropyrazol-3-one;
4,5-diamino-1-(2-hydroxyethyl)-2-methyl-1,2-dihydropyrazol-3-one;
4,5-diamino-2-(2-hydroxyethyl)-1-methyl-1,2-dihydropyrazol-3-one;
2,3-diamino-6,7-dihydro-1H,5H-pyrazolo[1,2-a]pyrazol-1-one;
2-amino-3-methylamino-6,7-dihydro-1H,5H-pyrazolo[1,2-a]pyrazol-1-one;
2-amino-3-dimethylamino-6,7-dihydro-1H,5H-pyrazolo[1,2-a]pyrazol-1-one;
2-amino-3-ethylamino-6,7-dihydro-1H,5H-pyrazolo[1,2-a]pyrazol-1-one;
2-amino-3-isopropylamino-6,7-dihydro-1H,5H-pyrazolo[1,2-a]pyrazol-1-one;
2-amino-3-(2-hydroxyethyl)amino-6,7-dihydro-1H,5H-pyrazolo[1,2-a]pyrazol-1-one;
2-amino-3-(2-hydroxypropyl)amino-6,7-dihydro-1H,5H-pyrazolo[1,2-a]pyrazol-1-one;
2-amino-3-bis(2-hydroxyethyl)amino-6,7-dihydro-1H,5H-pyrazolo[1,2-a]pyrazol-1-one;
2-amino-3-(pyrrolidin-1-yl)-6,7-dihydro-1H,5H-pyrazolo[1,2-a]pyrazol-1-one;
2-amino-3-(3-hydroxypyrrolidin-1-yl)-6,7-dihydro-1H,5H-pyrazolo[1,2-a]pyrazol-1-one;
2-amino-3-(piperidin-1-yl)-6,7-dihydro-1H,5H-pyrazolo[1,2-a]pyrazol-1-one;
2,3-diamino-6-hydroxy-6,7-dihydro-1H,5H-pyrazolo[1,2-a]pyrazol-1-one;
2,3-diamino-6-methyl-6,7-dihydro-1H,5H-pyrazolo[1,2-a]pyrazol-1-one;
2,3-diamino-6,6-dimethyl-6,7-dihydro-1H,5H-pyrazolo[1,2-a]pyrazol-1-one;
2,3-diamino-5,6,7,8-tetrahydro-1H,6H-pyridazino[1,2-a]pyrazol-1-one;
2,3-diamino-5,8-dihydro-1H,6H-pyridazino[1,2-a]pyrazol-1-one;
4-amino-5-dimethylamino-1,2-diethyl-1,2-dihydropyrazol-3-one;
4-amino-1,2-diethyl-5-ethylamino-1,2-dihydropyrazol-3-one;
4-amino-1,2-diethyl-5-isopropylamino-1,2-dihydropyrazol-3-one;
4-amino-1,2-diethyl-5-(2-hydroxyethylamino)-1,2-dihydropyrazol-3-one;
4-amino-5-(2-dimethylaminoethylamino)-1,2-diethyl-1,2-dihydropyrazol-3-one;
4-amino-5-[bis(2-hydroxyethyl)amino]-1,2-diethyl-1,2-dihydropyrazol-3-one;
4-amino-1,2-diethyl-5-(3-imidazol-1-yl-propylamino)-1,2-dihydropyrazol-3-one;
4-amino-1,2-diethyl-5-(3-hydroxypyrrolidin-1-yl)-1,2-dihydropyrazol-3-one;
4-amino-5-pyrrolidin-1-yl-1,2-diethyl-1,2-dihydropyrazol-3-one;
4-amino-5-(3-dimethylaminopyrrolidin-1-yl)-1,2-diethyl-1,2-dihydropyrazol-3-one; and
4-amino-1,2-diethyl-5-(4-methylpiperazin-1-yl)pyrazolidin-3-one.

The at least one oxidation base can be present in a total amount ranging from 0.001% to 20% by weight, for example from 0.005% to 10% by weight, and further for example from 0.01% to 5% by weight, relative to the total weight of the composition.

The at least one oxidation coupler according to the present disclosure may be chosen from benzene couplers, heterocyclic couplers, naphthalene couplers, and addition salts thereof.

By way of example, non-limiting mention may be made of benzene couplers that can be used in the compositions according to the present disclosure, including meta-aminophenols, meta-phenylenediamines, meta-diphenols, and addition salts thereof.

Among these oxidative couplers, mention may be made, in a non-limiting manner, of 2-methyl-5-aminophenol; 5-N-(β-hydroxyethyl)amino-2-methylphenol; 6-chloro-2-methyl-5-aminophenol; 3-aminophenol; 1,3-dihydroxybenzene; 1,3-dihydroxy-2-methylbenzene; 4-chloro-1,3-dihydroxybenzene; 2,4-diamino-1-(β-hydroxyethyloxy)benzene; 2-amino-4-(β-hydroxyethylamino)-1-methoxybenzene; 1,3-diaminobenzene; 1,3-bis(2,4-diaminophenoxy)propane; 3-ureidoaniline; 3-ureido-1-dimethylaminobenzene; sesamol; 1-β-hydroxyethylamino-3,4-methylenedioxybenzene; β-naphthol; 2-methyl-1-naphthol; 6-hydroxyindole; 4-hydroxyindole; 4-hydroxy-N-methylindole; 2-amino-3-hydroxypyridine; 6-hydroxybenzomorpholine; 3,5-diamino-2,6-dimethoxypyridine; 1-N-(β-hydroxyethyl)amino-3,4-methylenedioxybenzene; 2,6-bis(β-hydroxyethylamino)toluene; and the acid addition salts thereof.

The at least one oxidation coupler can be present in an amount ranging from 0.001 to 20% by weight, for example from 0.005% to 10% by weight, and further for example from 0.01% to 5% by weight, relative to the total weight of the composition.

In general, the addition salts of the oxidation bases and of the couplers that can be used in the context of the present disclosure can be, by way of non-limiting example, chosen from the addition salts with an acid, such as the hydrochlorides, hydrobromides, sulphates, citrates, succinates, tartrates, lactates, tosylates, benzenesulphonates, phosphates, and acetates; and the addition salts with a base, such as sodium hydroxide, potassium hydroxide, aqueous ammonia, amines, or alkanolamines.

The at least one fatty alcohol used in the composition of the present disclosure is chosen from long-chain fatty alcohols containing at least 20 carbon atoms, for example ranging from 20 to 30 carbon atoms, which may be linear or branched, saturated or unsaturated. For example, they may be primary fatty alcohols which have a linear and saturated chain. Further, by way of examples, non-limiting mention may be made of behenyl alcohol, arachidyl alcohol, lignoceryl alcohol, ceryl alcohol, montanyl alcohol, and mixtures thereof with a long chain containing at least 20 carbon atoms.

According to at least one embodiment of the present disclosure, the fatty alcohols that can be used in the context of the present disclosure are, by way of non-limiting example, a mixture of fatty alcohols comprised of 76% by weight of behenyl alcohol, 17% by weight of arachidyl alcohol, 1.5% by weight of lignoceryl alcohol, 5% by weight of stearyl alcohol, and 0.5% by weight of cetyl alcohol. Mixtures of this type are sold, for example, under the name NAFOL® 1822 C by the company Condea. In addition, non-limiting mention may also be made, for example of the following mixtures: the mixture sold under the name NAFOL® 2298 by the company Condea, which comprises 98% by weight of behenyl alcohol; the mixture sold under the name NAFOL® 20-22 by the company Condea, which comprises 30% by weight of behenyl alcohol, 58% by weight of arachidyl alcohol, and 6% by weight of lignoceryl alcohol; and the mixture sold under the name NAFOL® 20+ by the company Condea, which comprises 50% by weight of arachidyl alcohol, 29% by weight of behenyl alcohol, 14% by weight of lignoceryl alcohol, and 6% by weight of stearyl alcohol.

The at least one fatty alcohols containing at least 20 carbon atoms in the composition according to the present disclosure, may be present, in a total amount, ranging from 0.1% to 20% by weight, for example from 0.2% to 10% by weight, and further for example from 0.5% to 6% by weight, relative to the total weight of the composition.

The dye composition in accordance with the present disclosure may also further comprise at least one direct dye that may, for example, be chosen from nitrobenzene dyes, azo direct dyes, methine direct dyes, anthraquinone dyes, xanthene dyes, triarylmethane dyes, and addition salts thereof. These direct dyes may be nonionic, anionic, or cationic in nature.

The medium used in the compositions according to the present disclosure can be chosen from an aqueous medium or a medium comprising water and at least one organic solvent.

The at least one organic solvent used in the compositions according to the present disclosure may be chosen from monohydroxylated alcohols and polyols; wherein the monohydroxylated alcohols and polyols chosen as the at least one organic solvent are different from those which are chosen as the at least one fatty alcohol containing at least 20 carbon atoms in accordance with the present disclosure.

By way non-limiting example of monohydroxylated alcohols that can be used according to the present disclosure, mention may be made of C₁-C₄ lower alcohols such as ethanol, isopropanol, tert-butanol, or n-butanol, and mixtures thereof. In at least one embodiment of the present disclosure, the alcohol used is ethanol.

By way of non-limiting example of polyols that can be used according to the present disclosure, mention may be made of propylene glycol, polyethylene glycols, and glycerol. By way of non-limiting example of organic solvents, mention may also be made of polyol ethers such as 2-butoxyethanol, propylene glycol monomethyl ether, diethylene glycol monoethyl ether, and diethylene glycol monomethyl ether; and also aromatic alcohols such as benzyl alcohol or phenoxyethanol, and mixtures thereof.

The at least one organic solvent in the compositions according to of the present disclosure can be present, in a total amount, ranging from 0% to 30% by weight, for example from 0% to 20% by weight, relative to the total weight of the composition.

The compositions according to the present disclosure may further comprise at least one thickener, also referred to as a “rheology-adjusting agent”, wherein the thickener is different from the at least one nonionic derivative of cellulose with at least one hydrophobic substituent according to the present disclosure.

The rheology-adjusting agent, according to the present disclosure may be chosen from mineral or organic thickeners, for example polymeric associative thickeners; fatty alcohols different from the at least one fatty alcohol containing at least 20 carbon atoms in accordance with the present disclosure, such as oleyl alcohol; cellulosic derivatives other than the at least one nonionic derivative of cellulose with at least one hydrophobic substituent according to the present disclosure, such as hydroxyethylcellulose, hydroxypropylcellulose, and carboxymethylcellulose; and gums of microbial origin such as xanthan gum and scleroglucan gum.

The at least one thickener according to the present disclosure, can be present, in a total amount, ranging from 0.01% to 20% by weight, for example from 1% to 10% by weight, relative to the total weight of the composition.

The dye composition in accordance with the present disclosure may further comprise at least one adjuvant conventionally used in compositions for dyeing the hair.

As used herein, the term “adjuvant” is intended to mean at least one additive different from the above mentioned compounds, such as anionic, cationic, nonionic, amphoteric, or zwitterionic surfactants or mixtures thereof; non ionic, amphoteric, zwitterionic, anionic, or cationic polymers other than the at least one nonionic derivative of cellulose with at least one hydrophobic substituent according to the present disclosure, or mixtures of said polymers; penetrating agents; sequestering agents; fragrances; buffers; dispersants; conditioning agents such as, for example, modified or unmodified, volatile or non-volatile silicones; film-forming agents; ceramides; preservatives; opacifiers; vitamins; amino acids; oligopeptides; peptides; modified or unmodified, hydrolysed or nonhydrolysed proteins; enzymes; branched or unbranched fatty acids and alcohols; animal, plant or mineral waxes; hydroxylated organic acids; fatty alcohols other than the at least one fatty alcohol containing at least 20 carbon atoms in accordance with the present disclosure; UV screens; antioxidants and free-radical scavengers; antidandruff agents; seborrhoea-regulating agents; calmatives; mineral, plant or animal oils; polyisobutenes and poly(α-olefins); pigments; acids, bases, plasticizers, mineral fillers, pearlescent agents, flakes; antistatic agents; and reducing agents.

The at least one adjuvant as mentioned above may be present in an amount, for each of them, ranging from 0.01% to 40% by weight, for example from 0.1% to 25% by weight, relative to the total weight of the composition.

Those skilled in the art will take care to select this (or these) possible additional compound(s) in such a way that the beneficial properties intrinsically associated with the oxidation dyeing composition in accordance with the present disclosure are not, or not substantially, impaired by the addition(s) envisaged.

The pH of the dye composition in accordance with the present disclosure, by way of non-limiting example, ranges from 3 to 12, for example from 5 to 11. The pH may be adjusted to the desired value via at least one acidifying agent or basifying agent commonly used in the dyeing of keratin fibers or alternatively using at least one conventional buffer system.

Among the at least one acidifying agent, non-limiting mention may be made, by way of example, of mineral or organic acids such as hydrochloric acid, orthophosphoric acid, sulphuric acid, sulphonic acids, and carboxylic acids, for example acetic acid, tartaric acid, citric acid, and lactic acid.

Among the at least one basifying agents, non-limiting mention may, by way of example, be made of aqueous ammonia; alkali metal carbonates; alkanolamines such as mono-, di-, and triethanolamines and derivatives thereof; sodium hydroxide or potassium hydroxide; and the compounds of formula:

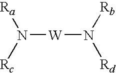

wherein:
W is a propylene residue optionally substituted with a hydroxyl group or a C₁-C₄ alkyl group;
Rₐ, R_{b}, R_{c}, and R_{d}, which may be identical or different, are chosen from hydrogen atoms, C₁-C₄ alkyl groups, and C₁-C₄ hydroxyalkyl groups.

The dye composition according to the present disclosure may be in various forms, such as in the form of creams or gels, or in any other form suitable for dyeing keratin fibers, including human hair.

The process for dyeing keratin fibers, of the present disclosure, is a process wherein the composition according to the present disclosure as defined above is applied to the fibers, in at least one embodiment, in the presence of at least one oxidizing agent for a period of time sufficient to develop the desired color. The color may be revealed at acidic, neutral, or alkaline pH and the at least one oxidizing agent may be added to the composition of the present disclosure just at the time of use, or it may be used starting from an oxidizing composition containing it, applied simultaneously with or sequentially to the composition of the present disclosure.

According to at least one embodiment, the composition according to the present disclosure is a ready-to-use composition, wherein the dyeing composition is mixed, for instance, at the time of use, with a composition comprising, in a medium suitable for dyeing, at least one oxidizing agent, the oxidizing agent being present in a sufficient amount to develop a coloration. The mixture obtained is subsequently applied to the keratin fibers. After a leave-on time ranging from 3 to 50 minutes, for example from 5 to 30 minutes, the keratin fibers are rinsed, washed with shampoo, rinsed again, and then dried.

The oxidizing agents conventionally used for the oxidation dyeing of keratin fibers are, for example, hydrogen peroxide; urea peroxide; alkali metal bromates; persalts such as perborates and persulphates; peracids; and oxidase enzymes, among which mention may be made of peroxidases, 2-electron oxidoreductases, such as uricases, and 4-electron oxygenases, such as laccases, these oxidoreductases being optionally combined with their customary cofactors, such as uric acid for uricases. In at least one embodiment of the present disclosure, the oxidizing agent is hydrogen peroxide.

The oxidizing composition may further comprise at least one adjuvant conventionally used in compositions for dyeing the hair, as defined above.

The pH of the oxidizing composition, comprising the at least one oxidizing agent, is such that, in one embodiment, after mixing with the dye composition, the pH of the resulting composition applied to the keratin fibers ranges from 3 to 12, for example from 5 to 10. The pH may be adjusted to the desired value via at least one acidifying agent or basifying agent normally used in the dyeing of keratin fibers, as defined above.

The ready-to-use composition which is finally applied to the keratin fibers may be in various forms, such as in the form of creams or gels, or in any other form suitable for dyeing keratin fibers, including human keratin fibers, such as the hair.

Another aspect of the present disclosure is a multicompartment dyeing device or dyeing “kit”, comprising at least one first compartment containing at least one dye composition as defined in the foregoing disclosure and at least one second compartment containing at least one oxidizing composition. This device may be equipped, by way of non-limiting example, with a mechanism for delivering the desired mixture to the hair, such as the devices described in French Patent Application FR A 2 586 913.

As disclosed herein, the percentages stated are by weight.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

The examples that follow are intended to illustrate the present disclosure without, however, being limiting in nature.

### EXAMPLES

### Composition 1

The following compositions were prepared; the amounts indicated are in grams unless otherwise indicated.

| | |
|---|---|
| Resorcinol | 0.685 |
| Stearic acid monoethanolamide | 4.8 |
| Oleic acid | 3 |
| Cetyl hydroxyethylcellulose (NATROSOL ® plus | 0.4 |
| grade 330CS sold by Aqualon/Hercules) | |
| Aqueous ammonia at 20% | 5 |
| Titanium dioxide | 0.3 |
| Ethanolamine | 0.8 |
| Oleth-10 | 1.8 |
| Aqueous solution containing 40% by weight of | 1.6 |
| Polyquaternium-6 (polydimethyldiallylammonium | |
| chloride, MERQUAT ® 100, sold by Ondeo/Nalco) | |
| m-Aminophenol | 0.14 |
| EDTA | 0.2 |
| 2,4-Diaminophenoxyethanol HCl | 0.02 |
| Aqueous solution containing 60% by weight of | 1.2 |
| hexadimethrine chloride | |
| (MEXOMERE ® PO sold by the company Chimex) | |
| Reducing agent | qs |
| Antioxidant | qs |
| Hydroxypropylmethylcellulose | 0.191 |
| Oleth-30 | 1.5 |
| Steareth-2 | 5.5 |
| C₂₀-C₂₂ alcohols (NAFOL ® 2022 EN sold by | 3 |
| the company Sasol) | |
| Toluene-2,5-diamine | 0.7623 |
| Water | Qs 100 |

### Application Protocol

Each composition was mixed, extemporaneously, with one and a half times its weight of an oxidizing composition having a pH in the region of 3 (aqueous hydrogen peroxide at 20 volumes) (6% by weight of H₂O₂). The mixture was easily prepared and had a good viscosity; it was easily applied to grey hair, comprising 90% white hairs, at a rate of 10 g per 1 g of hair, for 30 minutes. The hair was then rinsed, washed with a standard shampoo, and dried.

The hair coloration was evaluated visually. A hair coloration in a chestnut shade with a mahogany red tint was thus obtained.

The colorations had good properties, for instance, in terms of selectivity and strength. The compositions obtained were stable over time.

### Composition 2

The following compositions were prepared; the amounts given are in grams unless otherwise indicated.

| | |
|---|---|
| Stearic acid monoethanolamide | 4.8 |
| Oleic acid | 3 |
| Cetyl hydroxyethylcellulose (POLYSURF ® 67 sold | 0.5 TQ |
| by Aqualon/Hercules) | |
| p-Aminophenol | 0.24 |
| Aqueous ammonia at 20% | 5 |
| Titanium dioxide | 0.3 |
| Ethanolamine | 0.8 |
| 2-Amino-3-hydroxypyridine | 0.2 |
| 6-Hydroxyindole | 0.01 |
| Aqueous solution containing 40% by weight of | 1.6 |
| polyquaternium-6 (polydimethyldiallylammonium | |
| chloride, MERQUAT ®100, sold by Ondeo/Nalco) | |
| 4-Amino-2-hydroxytoluene | 0.24 |
| 2-Methyl-5-hydroxyethylaminophenol | 0.15 |
| m-Aminophenol | 0.024 |
| EDTA | 0.2 |
| 2,4-Diaminophenoxyethanol HCl | 0.02 |
| Aqueous solution containing 60% by weight of | 1.2 |
| hexadimethrine chloride | |
| (MEXOMERE ® PO sold by the company Chimex) | |
| Reducing agent | qs |
| Antioxidant | qs |
| Hydroxypropylmethylcellulose | 0.191 |
| Oleth-30 | 1.5 |
| PEG-40 stearate | 1.8 |
| Steareth-2 | 5.5 |
| Glyceryl lauryl ether | 0.5 |
| C20-C22 alcohols | 3 |
| (NAFOL ® 2022 EN sold by the company Sasol) | |
| Toluene-2,5-diamine | 0.2772 |
| Water | Qs 100 |

The application to the hair was carried out according to the protocol of Example 1. A hair coloration in a blond shade with a coppery mahogany tint was thus obtained.

The Following Compositions were Prepared (Amount Expressed in g %):

| | | Composition B | |
|---|---|---|---|
| | Compo- | (present | Compo- |
| | sition A | disclosure) | sition C |
| Ammonium hydroxide | 10 | 10 | 10 |
| Erythorbic acid | 0.50 | 0.50 | 0.50 |
| Ethanolamine | 0.70 | 0.70 | 0.70 |
| EDTA | 0.20 | 0.20 | 0.20 |
| Sodium sulfite | 0.50 | 0.50 | 0.50 |
| Titanium dioxide | 0.30 | 0.30 | 0.30 |
| 4-Amino-2-hydroxytoluene | 0.2460 | 0.2460 | 0.2460 |
| p-Phenylenediamine | 0.2160 | 0.2160 | 0.2160 |
| Stearylic alcohol | 2.64 | | |
| Mixture of linear alcohol | | 3 | 3 |
| C18/C20/C22/C24 7/58/30/6 | | | |
| (commercial name NAFOL ® | | | |
| 2022) | | | |
| Cetyl hydroxyethylcellulose | 0.40 | 0.40 | 1.40 |
| Oleic acid | 3 | 3 | 3 |
| Steareth-2 | 5.50 | 5.50 | 5.50 |
| Stearamide mea (96) (and) | 5 | 5 | 5 |
| Ethanolamine (2) (and) | | | |
| Stearic acid (2) | | | |
| Oleth-30 | 1.50 | 1.50 | 1.50 |
| Water | Qs 100 | Qs 100 | Qs 100 |

### Preparation of the Comparative Composition A

3 g % of NAFOL® 2022 having the following content C18/C20/C22/C24 7/58/30/6 comprised

### 0.21 g % of C18

1.74 g % of C20=0.0058 mole of CH₃(CH₂)₁₉OH
0.9 g % of C22=0.0027 mole of CH₃(CH₂)₂₁OH
0.18 g % of C24=0.0005 mole de CH₃(CH₂)₂₃OH.

The NAFOL® mixture contains 0.0090 mole of C20, C22, C24 alcohol. In the Composition A, the amount of alcohol with long chain was replaced by 0.0090 mole of C18 alcohol (stearylic alcohol) corresponding to 2.43 g.

To be comparable with the amount of C18 alcohol, 3 g % of NAFOL® 2022 was exchanged in the Composition A by 2.64 g % of stearylic alcohol (0.21+2.43 g).

Each composition was mixed, extemporaneously, with one and a half times its weight of an oxidizing composition (aqueous hydrogen peroxide at 20 volumes) (6% by weight of H2O2).

Each of the resulting mixtures was then applied onto locks of natural hair with 90% of white hair (BN) or permed hair with 90% of white hair (BP) with 15 g of composition for 1 g of hair. After 30 minutes, the hair was rinsed with water, washed with a standard shampoo, rinsed again, and dried.

### Colorimetric Evaluation

The color of the hair was determined by using the L*a*b* system, with a MINOLTA CM2002® spectrophotometer.

According to this system, L* indicates the lightness. The lower the value of L*, the more intense is the color of the hair. The chromaticity coordinates are expressed by the parameters a* and b*, a* indicating the axis of red/green shades and b* the axis of yellow/blue shades.

### Selectivity of the Coloration

The selectivity of the coloration is the variation of the color between natural colored hair and permed colored hair. Natural hair is representative of the nature of the hair at the root and the permed hair is representative of the nature of the hair at the tip.

The selectivity is measured by:

ΔE, which is the color variation between a natural colored lock and a permed colored lock, is obtained from the following formula:

$$Δ E = \sqrt{{( {L}^{*} - {L}_{o}^{*} )}^{2} + {( {a}^{*} - {a}_{o}^{*} )}^{2} + {( {b}^{*} - {b}_{o}^{*} )}^{2}}$$

wherein L* indicates lightness and a* and b* are the chromaticity coordinates of the natural colored locks whereas L₀* indicates the lightness and a₀* and b₀* are the chromaticity of the permed colored locks. The lower the value of ΔE, the lower the selectivity of the coloration is, and the more uniform the color is along the hair from the root to the tip.

The results are reported in the table below:

| Composition | Hair type | L* | a* | b* | ΔE |
|---|---|---|---|---|---|
| A | BN | 26.97 | 15.26 | −0.68 | 5.86 |
| | BP | 21.29 | 15.06 | 0.76 | |
| B (present | BN | 23.99 | 16.38 | 1 | 1.72 |
| disclosure) | BP | 23.01 | 16.1 | −0.39 | |
| C | BN | 26.50 | 16.48 | 0.33 | 5.70 |
| | BP | 20.91 | 15.48 | −0.16 | |

These results illustrate that the composition of the present disclosure exhibits a higher intensity of the color on the hair on natural hair. Furthermore, these results also illustrate an improvement of the selectivity, which indicates the homogeneity of the color along the fibers from the roots to the tips.

What is claimed is:

1. A dye composition for keratin fibers, comprising, in a medium suitable for dyeing:
(A) at least one nonionic derivative of cellulose comprising at least one hydrophobic substituent containing from 8 to 30 carbon atoms, present in an amount ranging from 0.1% to 1% by weight, relative to the total weight of the composition;
(B) at least one oxidation dye; and
(C) at least one fatty alcohol containing at least 20 carbon atoms.

2. A dye composition according to claim 1, wherein the at least one nonionic derivative of cellulose is chosen from hydroxyethylcelluloses substituted with at least one hydrophobic substituent containing from 8 to 30 carbon atoms.

3. A dye composition according to claim 1, wherein the at least one hydrophobic substituent is chosen from C₁₀-C₂₂ alkyl groups.

4. A dye composition according to claim 3, wherein the at least one hydrophobic substituent is chosen from cetyl groups.

5. A dye composition according to claim 1, wherein the degree of hydrophobic substitution ranges from 0.1% to 10% by weight, relative to the total weight of the polymer.

6. A dye composition according to claim 5, wherein the degree of hydrophobic substitution ranges from 0.4% to 0.8% by weight, relative to the total weight of the polymer.

7. A dye composition according to claim 1, wherein the at least one oxidation dye is chosen from oxidation bases, oxidation couplers, and addition salts thereof.

8. A dye composition according to claim 7, wherein the at least one oxidation base is chosen from para-phenylenediamines, bisphenylalkylenediamines, para-aminophenols, les bis-para-aminophenols, ortho-aminophenols, heterocyclic bases, and addition salts thereof.

9. A dye composition according to claim 7, wherein the at least one oxidation coupler is chosen from benzene couplers, heterocyclic couplers, naphthalene couplers, and addition salts thereof.

10. A dye composition according to claim 9, wherein the at least one benzene coupler is chosen from meta-aminophenols, meta-phenylenediamines, meta-diphenols, and addition salts thereof.

11. A composition according to claim 1, wherein the at least one fatty alcohol contains from 20 to 24 carbon atoms.

12. A composition according to claim 1, wherein the at least one fatty alcohol containing at least 20 carbon atoms is present in an amount ranging from 0.1% to 20% by weight, relative to the total weight of the composition.

13. A composition according to claim 12, wherein the at least one fatty alcohol containing at least 20 carbon atoms is present in an amount ranging from 0.2% to 10% by weight, relative to the total weight of the composition.

14. A dye composition according to claim 1, further comprising at least one direct dye chosen from nitrobenzene dyes, azo direct dyes, methine direct dyes, anthraquinone dyes, xanthene dyes, triarylmethane dyes, and addition salts thereof.

15. A dye composition according to claim 1, further comprising at least one oxidizing agent.

16. A dye composition according to claim 15, wherein the at least one oxidizing agent is hydrogen peroxide.

17. A process for the oxidation dyeing of keratin fibers, comprising,
applying to the fibers, in the presence of at least one oxidizing agent for a period of time sufficient to develop the desired color, a dye composition, comprising, in a medium suitable for dyeing:
(A) at least one nonionic derivative of cellulose comprising at least one hydrophobic substituent containing from 8 to 30 carbon atoms, in an amount ranging from 0.1% to 1% by weight, relative to the total weight of the composition;
(B) at least one oxidation dye; and
(C) at least one fatty alcohol containing at least 20 carbon atoms.

18. A multicompartment device, comprising
at least one first compartment containing at least one dye composition comprising, in a medium suitable for dyeing:
(A) at least one nonionic derivative of cellulose comprising at least one hydrophobic substituent containing from 8 to 30 carbon atoms, present in an amount ranging from 0.1% to 1% by weight, relative to the total weight of the composition;
(B) at least one oxidation dye; and
(C) at least one fatty alcohol containing at least 20 carbon atoms; and
at least one second compartment containing at least one oxidizing agent.
